(19) United States
(12) Patent Application Publication
(10) Pub. No.: US 2005/0137554 A1
(43) Pub. Date: Jun. 23, 2005

(54) Interlabial pad and individual wrapping container

(75) Inventors: Satoshi Mizutani, Kagawa (JP); Wataru Yoshimasa, Kagawa (JP); Yuki Noda, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-Shi (JP)

(21) Appl. No.: 11/007,660

(22) Filed: Dec. 8, 2004

(30) Foreign Application Priority Data
Dec. 9, 2003 (JP) 2003-411083

(51) Int. Cl. A61F 13/15; A61F 13/20

(52) U.S. Cl. 604/385.02

(57) ABSTRACT

An interlabial pad and individual wrapping container is provided capable of reducing an incidence rate of defective products by preventing the interlabial pad from shifting its proper position on the individual wrapping container during the step of packing in a product line. An individual wrapping container for individually packing an interlabial pad applied by inserting into wearer's labia pudendi, the individual wrapping container including an inner face having a region to be brought into contact with the interlabial pad when the interlabial pad is included therein, and an outer face to be the reverse face of the same, wherein the inner face is provided with a displacement preventive means to bind the interlabial pad at a predetermined position thereby preventing displacement of the interlabial pad.

### CROSS-REFERENCE TO RELATED APPLICATIONS

This application is based upon and claims the benefit of priority from Japanese Patent application No. 2003-411083 filed on Dec. 9, 2003, the entire contents of which is incorporated herein by reference.

### FIELD OF THE INVENTION

The present invention relates to an interlabial pad applied between female labia, and an individual wrapping container for wrapping an interlabial pad. More particularly, the invention relates to an individual wrapping container which prevents the interlabial pad from getting out of its proper position.

### RELATED ART

Conventionally, interlabial pads which absorb bodily fluids such as menstrual blood or urine through applying to female labia have been known as absorptive articles for use in menstruation or in defense against urinary incontinence, or both of the same. Because such an interlabial pad achieves closer contact with the body in comparison with sanitary napkins, spreading of the menstrual blood to be brought into contact with the body on a wider area can be prevented. Therefore, it is sanitary as well as clean. Further, the interlabial pad is more compact than a sanitary napkin, feel during application is excellent and comfortable, and is also characterized in that the interlabial pad leads to less psychological resistance upon application, compared to tampons that are inserted into vagina.

Examples of such interlabial pad include an interlabial pad (trade name, “Envive (Moderate)”) subjected to test marketing by the Procter & Gamble (hereinafter, referred to as PG) (for example, JP-T-11-500341 (the term “JP-T” as used herein means a published Japanese translation of a PCT application) hereinafter, referred to as “Patent Document 1”). This interlabial pad has a liquid permeable surface side sheet, a liquid impermeable back face side sheet, and an absorptive member disposed between the surface side sheet and the back face side sheet. An emollient agent (skin softening agent) to elevate conformability is included in the surface side sheet of this interlabial pad for improving the conformability to the inner wall of the labia during its application. Such an interlabial pad containing the emollient agent is folded in two such that the back face side sheet is opposed with each other, and packed such that the surface side sheet and the inner face of the individual wrapping container are opposed.

However, due to the emollient agent included in the surface side sheet, the face where the surface side sheet of the interlabial pad is brought into contact with the inner face of the individual wrapping container is liable to slip. Thus, in the step of packing the interlabial pad, the interlabial pad becomes liable to shift from its proper position on the individual wrapping container, thereby resulting in probability of production of a defective product upon manufacture of the interlabial pad.

The present invention was made in view of the problems as described above, and an object thereof is to provide an interlabial pad and an individual wrapping container capable of reducing an incidence rate of defective products of the completed products by preventing shifting of the interlabial pad from its proper position on the individual wrapping container during the step of packing in the product line.

### SUMMARY OF THE INVENTION

More specifically, the present invention provides the following matters.

(1) An individual wrapping container for individually wrapping an interlabial pad applied by inserting into wearer's labia; wherein the individual wrapping container comprises an inner face having a region to be brought into contact with the interlabial pad when the interlabial pad is included therein, and an outer face to be the reverse face of the inner face; and wherein the inner face comprising a displacement preventive means to bind the interlabial pad at a predetermined position thereby preventing the displacement of the interlabial pad. The aforementioned “displacement” refers to shifting of the interlabial pad from a proper position, where the interlabial pad is placed originally in the wrapping step, on the individual wrapping container. Furthermore, “bind” refers to restraining movements of the interlabial pad within the individual wrapping container, or stopping the movements of the interlabial pad. Moreover, “inner face of the individual wrapping container” represents a face to be brought into contact with the interlabial pad. In other words, when the outer face of the individual wrapping container has a region which is brought into contact with the interlabial pad by, for example, folding of the individual wrapping container material, the individual wrapping container material of such a region shall be a part of the inner face of the individual wrapping container.

The individual wrapping container of the invention has a displacement preventive means for preventing the displacement of the interlabial pad, by binding the interlabial pad on the inner face. Accordingly, the position of the interlabial pad placed on the individual wrapping container in the wrapping step can be preserved as it is, therefore, incidence rate of the defectives of the product resulting from disposing at an improper position can be reduced. In addition, because displacement of the interlabial pad included in the individual wrapping container is prevented in the packed state, deformation or damage caused by movement of the interlabial pad within the individual wrapping container can be prevented in the traveling step as well. Thus, foreign body sensation posed to the wearer by the altered shape, raising of naps on the outer face of the interlabial pad resulting from friction created on the outer face of the interlabial pad, and the like can be prevented.

For reference, this displacement preventive means does not prevent the wearer from readily drawing the interlabial pad from the individual wrapping container without sense of resistance. In addition, the individual wrapping container may pack a part of the interlabial pad, or may pack such that it wraps the entirety. Further, the interlabial pad may have a flat shape, cylindrical shape, or any shape such as sphere or cube, as long as it can be packed in the individual wrapping container.

(2) The individual wrapping container according to (1), wherein the displacement preventive means has a shifting resitant element having a coefficient of static friction of a range from 0.200 to 0.600 disposed on the entire face of the inner face, or on a part of the inner face corresponding to the entirety or a part of the predetermined position.

The “coefficient of static friction” is measured as a ratio of a force of one substance which is going to slip on a contact face with other substance, and a force acting on a tangential direction of the two substances as a force to inhibit the force exerted when the substance is going to slip, when two stationary substances being in contact with each other are inclined. Specifically, the coefficient of static friction of the inner face of the individual wrapping container is measured as a ratio of a force of the inner face of the individual wrapping container which is going to slip on a glass plate, and a force of the inner face of the individual wrapping container pushing against the glass plate, when the glass plate is in contact with the inner face of the individual wrapping container, and the glass plate is fixed.

According to the displacement preventive means of the invention, a shifting resitant element is provided on at least a predetermined position on the inner face of the individual wrapping container, and this shifting resitant element has a coefficient of static friction of a range form 0.200 to 0.600. The “shifting resitant element disposed on the entirety or a part” herein refers to any case including a case when the material constituting the individual wrapping container constitutes the entirety or a part of the inner face of the individual wrapping container as the shifting resitant element.

Examples of the material of the shifting resitant element having a coefficient of static friction of a range from 0.200 to 0.600 provided on the inner face of the individual wrapping container include polyethylene, polypropylene, polyethylene terephthalate, polyvinyl alcohol, polylactic acid, polybutyl succinate, or nonwoven fabrics, paper, and laminate materials thereof having a thickness of 15 to 60 μm. Alternatively, a breathable film obtained by mixing an inorganic filler to resin and drawing the mixture may be used.

Specifically, examples include films prepared using a low density polyethylene (LDPE) resin as a main component and adjusted to give a specific weight per unit area in a range from 15 to 35 g/m² with fine convexities provided thereon. Height of the fine convexity is in a range from 0.05 mm to 2 mm, and interval between the fine convexities is in a range from 0.3 mm to 20 mm. Among them, films prepared using a low density polyethylene (LDPE) resin as a main component to have fine convexities arranged with the height of the convexity being 0.2 mm, the interval therebetween being 1.5 mm and the shape being staggered have a coefficient of static friction of 0.250. Further, a rubber component which can provide an elastomer characteristic, an anti-slip agent or the like may be admixed together with the low density polyethylene (LDPE) resin. Alternatively, examples include materials for the individual wrapping container prepared by spraying the melt blown on a film comprising polyethylene, polypropylene or the like, or a nonwoven fabric or paper to have a melt blown surface on the inner face of the individual wrapping container. Because the melt blown is an aggregate of very fine fibers, a great contact area is achieved. Accordingly, the coefficient of static friction falling within a suitable range can be achieved. In addition, it is preferred that the individual wrapping container can block the color of the bodily fluid absorbed in the interlabial pad. Thus, a pigment may be admixed in a range from 0.2 to 10%, or printing with an ink or the like may be performed.

(3) The individual wrapping container according to (1) wherein the displacement preventive means is a breathable material constituting the individual wrapping container, and the air permeability of the breathable material is a range form 0.040 KPa·s/m to 1.000 KPa·s/m.

The “air permeability” referred to above means a degree of circulation of the air passing through the individual wrapping container.

The individual wrapping container of the invention has breathability in a through-thickness direction, and in connection with the breathability, the air permeability is in a range from 0.040 KPa·s/m to 1.000 KPa·s/m. When the air permeability of the individual wrapping container is less than 0.040 KPa·s/m, the interlabial pad can not be fixed without fail on the individual wrapping container by a suction apparatus via the individual wrapping container during feeding while suctioning with the suction apparatus in the packing step of the interlabial pad for covering the interlabial pad with the individual wrapping container. Thus, due to the external pressure such as wind velocity during the production, the interlabial pad becomes liable to be shifted from the position where it is originally placed. On the other hand, when the air permeability is greater than 1.000 KPa·s/m, foreign substances such as dust are liable to attach on the interlabial pad through the individual wrapping container while carrying. In addition, the interlabial pad can be kept to be sanitary through being drawn by the wearer without touching the surface side sheet of the interlabial pad to which foreign substances such as dust are attached, and thus, the interlabial pad can be readily worn without sense of resistance. However, breathability is so high that the surface side sheet is at risk of being damaged by pressure exerted by suction with the suction apparatus.

Examples of the material of the individual wrapping container having an air permeability of the range from 0.040 KPa·s/m to 1.000 include films having communicating pores obtained by mixing an inorganic filler to a resin and drawing the mixture, or by partially subjecting to a treatment for opening pores, e.g., polyethylene, polypropylene, polyethylene terephthalate, polyvinyl alcohol, polylactic acid, polybutyl succinate, or nonwoven fabrics, paper, and laminate materials thereof having a thickness of 15 to 60 μm and having communicating pores provided therein. Specifically, so called SMS nonwoven fabrics are included constituted from spun bond/melt blown/spun bond with the specific weight per unit area of 6 to 10 gsm/2 to 20 gsm/6 to 10 gsm. Because the melt blown is an aggregate of very fine fibers, it has a high density and excellent barrier property. Therefore, adequate air permeability is achieved by adjusting the specific weight per unit area or the like of the melt blown layer. Specifically, an SMS nonwoven fabric constituted from spun bond/melt blown/spun bond with the specific weight per unit area of 7 gsm/3 gsm/7 gsm has an air permeability of 0.074 Kpa·s/m.

(4) The individual wrapping container according to (1); wherein the displacement preventive means is a sheet piece formed by being mounted on the inner face or the outer face or by extending a part of a material that constitutes the individual wrapping container; and wherein the sheet piece has length and flexibility enabling to wind around a part or whole of the interlabial pad, and has a release part permitting easy release of the winding when the interlabial pad is drawn from the a individual wrapping container.

The aforementioned “wind around a part or whole” refers to passing out of view of a part of the interlabial pad from outside of the sheet piece, or passing out of view of whole of the interlabial pad from outside of the sheet piece by wrapping of the interlabial pad with the sheet piece. Further, the release part is positioned such that it can be seen with eyes, and the binding of the interlabial pad can be released by pulling the interlabial pad, or pulling this release part.

The individual wrapping container of the invention has a sheet piece capable of winding around a part or whole of the interlabial pad such that the interlabial pad is bound, as a displacement preventive means, which is mounted on the inner face or the outer face of the individual wrapping container. Accordingly, the sheet piece encases the circumference of the site of the interlabial pad where particularly hypersensitive clitoris or anus is liable to be in contact, therefore, the site can be protected from the impact while carrying on behalf of the buckling strength of the sheet piece, thereby preventing the wearer's foreign body sensation and damage. Moreover, because displacement of the interlabial pad included in the individual wrapping container is prevented in the packed state, shape of the interlabial pad within the individual wrapping container can be preserved. Thus, an interlabial pad enabling to prevent wearer's foreign body sensation due to the altered shape, and damage such as raising of naps on the outer face of the interlabial pad resulting from friction created on the outer face of the interlabial pad can be provided.

The sheet piece may be directly mounted on the inner face or the outer face of the individual wrapping container, or may be formed by extending the marginal part of the individual wrapping container in part. In addition, the release part of the sheet piece can be a portion having perforations provided for the purpose of enabling cleavage, or a portion to which a pressure-sensitive adhesive is applied allowing easy detachment between the inner face of the individual wrapping container and the adhered face. Alternatively, when the interlabial pad is bound through merely being wound by the sheet piece, the release part may be a portion for unfolding this winding (for example, end of the sheet piece), or when the sheet piece itself comprises a material which can be readily cleaved, the release part may be any portion of the sheet piece.

(5) The individual wrapping container according to (4) wherein the individual wrapping container has an opening part for opening the individual wrapping container, and wherein the release part of the sheet piece is disposed in the vicinity of the opening part of the individual wrapping container.

The individual wrapping container of the invention has an opening part for opening the individual wrapping container, and the sheet piece has a release part for releasing the binding to the interlabial pad, with the release part being provided on the side of the opening part. Thus, the release part of the sheet piece can be seen with eyes concurrently with opening of the individual wrapping container. Accordingly, the wearer can readily draw the interlabial pad from the individual wrapping container without any sense of resistance.

(6) An interlabial pad applied by inserting into wearer's labia, which comprises a surface side sheet orientated to a body face side and a back face side sheet orientated to the reverse side face of the body face side, wherein whole or a part of the surface side sheet and/or back face side sheet has a displacement preventive means for preventing shifting from a predetermined position on the individual wrapping container where the interlabial pad is disposed when the interlabial pad is packed in an individual wrapping container.

The interlabial pad of the invention has a displacement preventive means on the surface side sheet and/or back face side sheet. Displacement of the interlabial pad placed on the individual wrapping container is thereby prevented during the production, therefore, incidence rate of the defective products of the completed product can be reduced. In addition, because displacement of the interlabial pad included in the individual wrapping container is prevented in the packed state, shape of the interlabial pad within the individual wrapping container can be preserved. Thus, an interlabial pad enabling to prevent wearer's foreign body sensation or damage can be provided.

The interlabial pad of the present invention can be also used for absorbing non menstrual vaginal discharges and for urinary incontinence, in addition to use in absorbing menstrual blood.

(7) The interlabial pad according to (6) wherein the displacement preventive means of the interlabial pad comprises a shifting resitant element having a coefficient of static friction of a range from 0.200 to 0.600.

The coefficient of static friction of the aforementioned interlabial pad is measured as a ratio of a force of the interlabial pad which is going to slip on a glass plate, and a force of the interlabial pad pushing against the glass plate, when the glass plate is in contact with the interlabial pad, and the glass plate is fixed.

According to the interlabial pad of the present invention, the displacement preventive means is disposed on the surface side sheet when the surface side sheet is in contact with the individual wrapping container; while an shifting resitant element having a coefficient of static friction of a range of 0.200 to 0.600 is disposed on the back face side sheet when the back face side sheet is in contact with the individual wrapping container.

The shifting resitant element is, specifically, as described below. That is, as a material for the surface side sheet, pulp, chemical pulp, rayon, cotton, acetate, a synthetic fiber, physically embossed rayon or acetate, chemical pulp crosslinked with a crosslinking agent followed by crimping may be used alone or in combination. Examples of the method of the production of the sheet include a spun bond method, a point bond method, an air through method, a melt blown method, a spun lace method, a paper making method and the like. Among such methods of the production, the spun lace method is preferred in which fiber confounding is executed by hydroentanglement, thereby capable of eliminating an oil solution attached to the fiber by the water flow, because any oil solution, lubricant or the like which may elevate the slipping property with the inner face of the individual wrapping container is not preferably included in the outer face of the interlabial pad.

On the other hand, examples of the material for the back face side sheet include polyethylene, polypropylene, polyethylene terephthalate, polyvinyl alcohol, polylactic acid, polybutyl succinate, or nonwoven fabrics, paper, and laminate materials thereof having a thickness of 15 to 60 μm. Alternatively, a breathable film obtained by mixing an inorganic filler to a resin and drawing the mixture may be also used. Specifically, examples include films prepared using a low density polyethylene (LDPE) resin as a main component and adjusted to give a specific weight per unit area in a range from 15 to 35 g/m² with fine convexities provided thereon. Height of the fine convexity is in a range from 0.05 mm to 2 mm, and interval between the fine convexities is in a range from 0.3 mm to 20 mm. Among them, films prepared using a low density polyethylene (LDPE) resin as a main component to have fine convexities arranged with the height of the convexity being 0.2 mm, the interval therebetween being 1.5 mm and the shape being staggered have a coefficient of static friction of 0.250. Further, a rubber component which can provide an elastomer characteristic, an anti-slip agent or the like may be admixed together with the low density polyethylene (LDPE) resin. Alternatively, examples include back face side sheets prepared by spraying the melt blown on a film comprising polyethylene, polypropylene or the like, or a nonwoven fabric or paper to have a melt blown surface on the outer face of the interlabial pad. Because the melt blown is an aggregate of very fine fibers, a great contact area is achieved. Accordingly, the coefficient of static friction falling within a suitable range can be achieved.

(8) A wrapping body which comprises an interlabial pad applied by inserting into wearer's labia, and an individual wrapping container for individually wrapping the interlabial pad, wherein a coefficient of static friction of a part where the interlabial pad is in contact with the individual wrapping container is in a range from 0.220 to 0.500.

The coefficient of static friction at a part where the interlabial pad is in contact with the individual wrapping container is measured as a ratio of a force of the outer face of the interlabial pad which is going to slip on the inner face of the individual wrapping container, and a force of the outer face of the interlabial pad pushing against the inner face of the individual wrapping container, when the outer face of the interlabial pad is in contact with the inner face of the individual wrapping container, and the interlabial pad is fixed on the inner face of the individual wrapping container.

According to the wrapping body of the invention, it is preferred that the coefficient of static friction between the outer face of the interlabial pad, i.e., the surface side sheet or back face side sheet, and the inner face of the individual wrapping container is in a range from 0.220 to 0.500. When the coefficient of static friction of the inner face of the individual wrapping container is less than 0.200, or the coefficient of static friction of the outer face of the interlabial pad is less than 0.200, the coefficient of static friction between the inner face of the individual wrapping container and the outer face of the interlabial pad tends to be less than 0.220. Further, when the coefficient of static friction of the inner face of the individual wrapping container is greater than 0.600, or when the coefficient of static friction of the outer face of the interlabial pad is greater than 0.600, the coefficient of static friction between the outer face of the interlabial pad and the inner face of the individual wrapping container tends to be greater than 0.500. Specific examples are as follows. The inner face of the individual wrapping container is a film comprising a low density polyethylene (LDPE) resin as a main component and having fine convexities arranged with the height of the convexity being 0.2 mm, the interval therebetween being 1.5 mm and the shape being staggered, with the coefficient of static friction of which being 0.250. On the other hand, the outer face of the interlabial pad, i.e., the surface side sheet, is 35 g/m² spun lace of 100% rayon fiber, with the coefficient of static friction of which being 0.445. The coefficient of static friction when contact faces of respective sheets are faced is 0.299.

(9) A wrapping body which comprises an interlabial pad applied by inserting into wearer's labia, and an individual wrapping container for individually wrapping the interlabial pad, wherein a connecting means for connecting a part of the interlabial pad to a part of the individual wrapping container for preventing a displacement of the interlabial pad by binding the interlabial pad at a predetermined position on the individual wrapping container.

In the wrapping body of the invention, the interlabial pad and the individual wrapping container may not be completely separated, with a part of the interlabial pad being connected to a part of the individual wrapping container. Thus, movement of the interlabial pad included in the individual wrapping container followed by remaining at an inadequate position is prevented in the packed state, shape of the interlabial pad within the individual wrapping container can be preserved. Accordingly, an interlabial pad enabling to prevent wearer's foreign body sensation due to the altered shape of the interlabial pad and individual wrapping container, and damage such as raising of naps of the interlabial pad resulting from friction created on the outer face of the interlabial pad can be provided.

(10) The wrapping body according to (9) wherein the connecting means integrally connects a part of the individual wrapping container to a part of the interlabial pad.

The “integrally connect” as described above refers to joining such that a part of the interlabial pad is integrated with a part of the individual wrapping container, for example, binding by way of a concavity and convexity part of engaging emboss in cases of joining under a condition of emboss not to result in thermal deposition may be included.

According to the wrapping body of the invention, shape of the interlabial pad within the individual wrapping container can be preserved because displacement of the interlabial pad included in the individual wrapping container can be prevented by connection of a part of the individual wrapping container to a part of the interlabial pad.

(11) The wrapping body according to (9) or (10) wherein a part connected by the connecting means or the vicinity thereof has a cutoff part to be cut off when the interlabial pad is drawn from the individual wrapping container.

The “cutoff part” described above refers to a part permitting cutting at a portion where a part of the interlabial pad and a part of the individual wrapping container are joined to separate the two, which may be, for example, perforations.

The wrapping body of the invention has a cutoff part which is cut off at a connecting means where the interlabial pad had been fixed within the individual wrapping container. Accordingly, perforations are provided at the connected part, for example, at a portion of the interlabial pad in cases of engaged emboss, and thus, the interlabial pad can be cut to separate from the individual wrapping container by merely cutting off along the perforations, when the interlabial pad is drawn from the individual wrapping container. Therefore, the wearer can readily draw the interlabial pad from the individual wrapping container without any sense of resistance.

(12) The wrapping body according to (9) wherein the connecting means is an adhesive applied for adhering the interlabial pad and/or the individual wrapping container.

According to the wrapping body of the invention, the connecting means may be an adhesive. When the interlabial pad is drawn from the individual wrapping container, fibers to which the adhesive is adhered are cut to separate from the interlabial pad at the portion of the interlabial pad which is an aggregate of fibers. Thus, when the interlabial pad is drawn from the individual wrapping container, a part of the fibers to which the adhesive is adhered is cut to separate by pulling the interlabial pad body. Accordingly, the wearer can readily draw the interlabial pad from the individual wrapping container. Furthermore, a portion of the interlabial pad comprising a fiber aggregate or a film material may be subjected to a treatment for detachment by e.g., applying silicon or the like. By pulling the interlabial pad body, the interlabial pad can be drawn without damage thereto while leaving the adhesive on the individual wrapping container. In connection with the place where the adhesive is applied, it is preferably applied to a region exposed out from the labia, taking into account of the foreign body sensation to the wearer.

[Advantage of the Invention]

As described hereinabove, the present invention can provide an individual wrapping container and an interlabial pad capable of reducing an incidence rate of defective products by preventing the shifting of the interlabial pad from its intended position to be placed on the individual wrapping container during the step of packing the interlabial pad, and further, capable of preventing deformation or damage caused by movement of the interlabial pad within the individual wrapping container in the packed state.

### BRIEF DESCRIPTION OF THE DRAWINGS

FIG. 1 is a perspective view illustrating a wrapping body according to first embodiment of the present invention.

FIG. 2 is a view illustrating a step for producing a wrapping body according to second embodiment of the invention.

FIG. 3 is a view illustrating an opened state of a wrapping body according to third embodiment of the invention.

FIG. 4 is a cross sectional view illustrating the wrapping body according to the third embodiment of the invention.

FIG. 5 is a view illustrating an opened state of an individual wrapping container according to the third embodiment of the invention.

FIG. 6 is a view illustrating the opened state of the individual wrapping container according to the third embodiment of the invention.

FIG. 7 is an end view illustrating a wrapping body according to fourth embodiment of the invention.

FIG. 8 is an end view illustrating a wrapping body according to fifth embodiment of the invention.

FIG. 9 is an end view illustrating a wrapping body according to sixth embodiment of the invention.

FIG. 10 is a cross sectional view illustrating the wrapping body according to the sixth embodiment of the invention.

FIG. 11 is a view illustrating the process for measuring coefficient of static friction according to the invention.

FIG. 12 is a view illustrating the process for measuring coefficient of static friction according to the invention.

### DESCRIPTION OF THE PREFERRED EMBODIMENT

Hereinafter, each embodiment of the present invention is explained with reference to drawings. In the following description of the embodiment, the identical reference numeral is assigned for the identical constituent element to omit or simplify the explanation thereof. [First Embodiment]

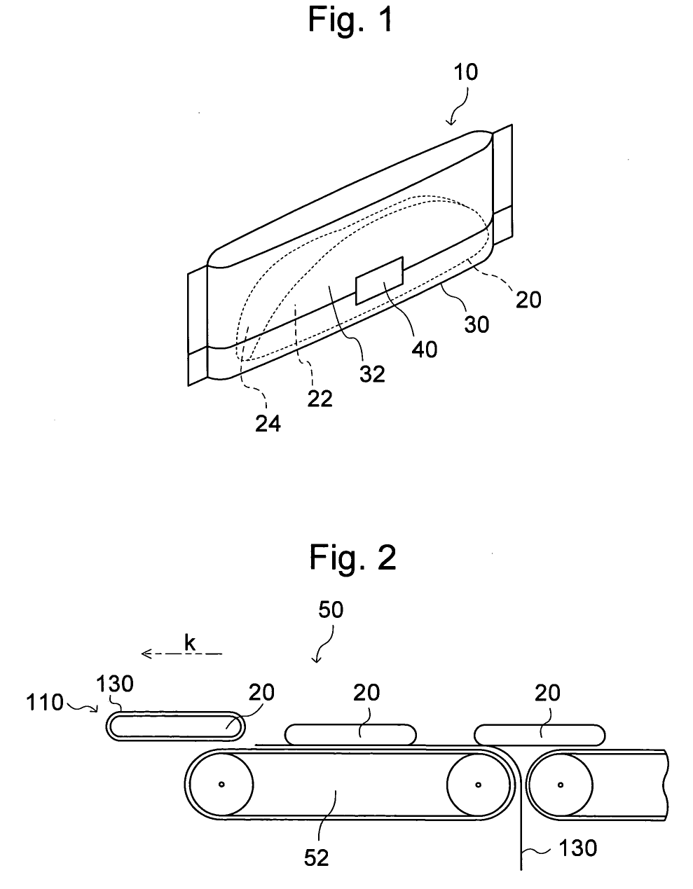

FIG. 1 presents a perspective view of a wrapping body 10 according to first embodiment of the invention.

As is shown in FIG. 1, the wrapping body 10 has an interlabial pad 20 and an individual wrapping container 30, with the individual wrapping container 30 having an opening part 40.

The interlabial pad 20 has a surface side sheet 22 and a back face side sheet 24, is folded in two such that the back face side sheet 24 is opposed with each other, and is packed in the individual wrapping container 30 such that the surface side sheet 22 and the inner face of the individual wrapping container 30 are opposed. The interlabial pad 20 may be folded in two such that the surface side sheet 22 is opposed with each other, and packed in the individual wrapping container 30 such that the back face side sheet 24 and the inner face of the individual wrapping container 30 are opposed.

At a predetermined position of the inner face of the individual wrapping container 30 which is brought into contact with the surface side sheet 22 or the back face side sheet 24 of the interlabial pad 20, a shifting resitant element adjusted such that the coefficient of static friction becomes a range from 0.200 to 0.600 is disposed. Furthermore, the coefficient of static friction between the surface side sheet 22 or back face side sheet 24 of the interlabial pad 20 to be brought into contact with the individual wrapping container 30, and the inner face of the individual wrapping container 30 to be brought into contact with the outer face of the interlabial pad 20 is in a range from 0.220 to 0.500. Thus, by adjusting the coefficient of static friction of each member, the interlabial pad 20 can be bound in the step of production, or in the state of the wrapping body 10, enabling to prevent displacement of the interlabial pad 20. In addition, because each member has a proper coefficient of static friction, the wearer can readily draw the interlabial pad 20 from the individual wrapping container without any sense of resistance.

Although a shifting resitant element having a coefficient of static friction of the range from 0.200 to 0.600 is disposed at a predetermined position on the inner face of the individual wrapping container 30 according to the first embodiment, the shifting resitant element having a coefficient of static friction of 0.200 to 0.600 may be disposed at a predetermined position on the surface side sheet 22 or the back face side sheet 24 of the interlabial pad 20. [Second Embodiment]

FIG. 2 is a view illustrating a step for producing the wrapping body 110 according to second embodiment of the invention.

This embodiment is different from the above first embodiment in terms of the shifting resitant element rendered to have the air permeability of the individual wrapping container 130 in a through-thickness direction. The air permeability of the individual wrapping container 130 is a range from 0.040 KPa·s/m to 1.000 KPa·s/m. FIG. 2 shows a step for producing the wrapping body 110. First, a suction apparatus 52 is provided in a product line 50, and on the upper face of this suction apparatus 52 is placed the individual wrapping container 130. Next, the interlabial pads 20 are placed on the individual wrapping container 130 at regular intervals, and fed in a direction represented by the symbol k. At this time, suction via the individual wrapping container 130 having an air permeability of 0.040 KPa·s/m to 1.000 KPa·s/m results in binding of the interlabial pad 20 on the suction apparatus 52. The wrapping body 110 thereby can be readily formed, and production of defective products can be prevented. [Third embodiment]

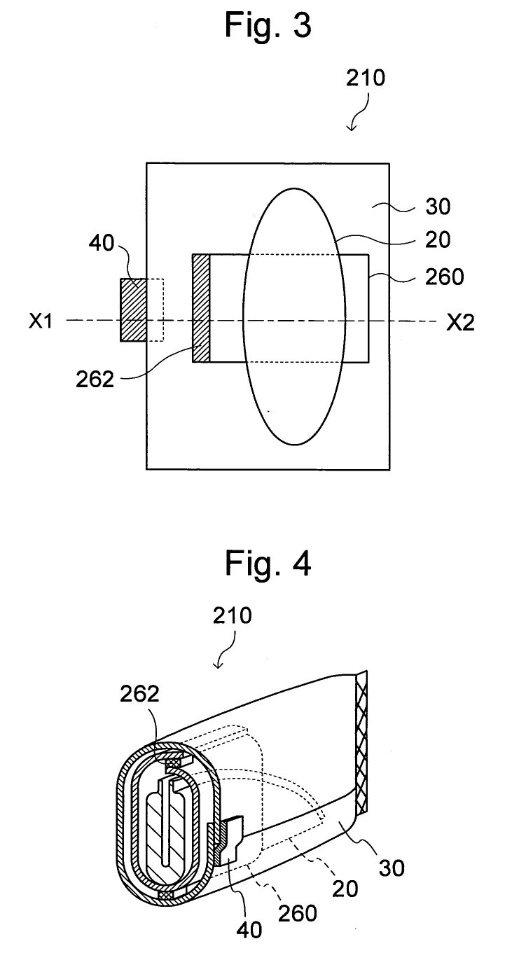

FIG. 3 is a view illustrating an opened state of the wrapping body 210 in relation to the third embodiment of the invention. FIG. 4 depicts a cross sectional view in a direction of X1-X2 following assembly of the wrapping body 210 according to the third embodiment of the invention.

This embodiment is different from the above first embodiment, as shown in FIG. 3, in terms of a shifting resitant element being a sheet piece 260. The sheet piece 260 is provided between the interlabial pad 20 and the individual wrapping container 30. Further, the sheet piece 260 has length and flexibility enabling winding at least a part or whole of the interlabial pad 20. There is the possibility of irritating to the wearer's skin at the portion bent and folded when the interlabial pad is bent and folded while carrying. However, as in this embodiment, encasing of the circumference of the site of the interlabial pad 20 where particularly hypersensitive clitoris or anus is liable to be in contact, through the use of the sheet piece 260, protects from the impact while carrying on behalf of the buckling strength of the sheet piece 260, thereby preventing the wearer's foreign body sensation and damage. Moreover, because displacement of the interlabial pad 20 included in the individual wrapping container 30 is prevented, shape of the interlabial pad 20 within the individual wrapping container 30 can be preserved. Thus, wearer's foreign body sensation due to the altered shape, and damage such as raising of naps on the outer face of the interlabial pad 20 resulting from friction created on the outer face of the interlabial pad 20 can be prevented. In addition, the sheet piece 260 has a release part 262 for stripping off the interlabial pad 20, while the individual wrapping container 30 has an opening part 40 for opening the individual wrapping container 30, with the release part 262 being provided on the side of the opening part 40. Accordingly, the release part 262 of the sheet piece 260 can be seen with eyes concurrently with opening of the individual wrapping container 30. Therefore, the wearer can readily draw the interlabial pad 20 from the individual wrapping container 30 without any sense of resistance.

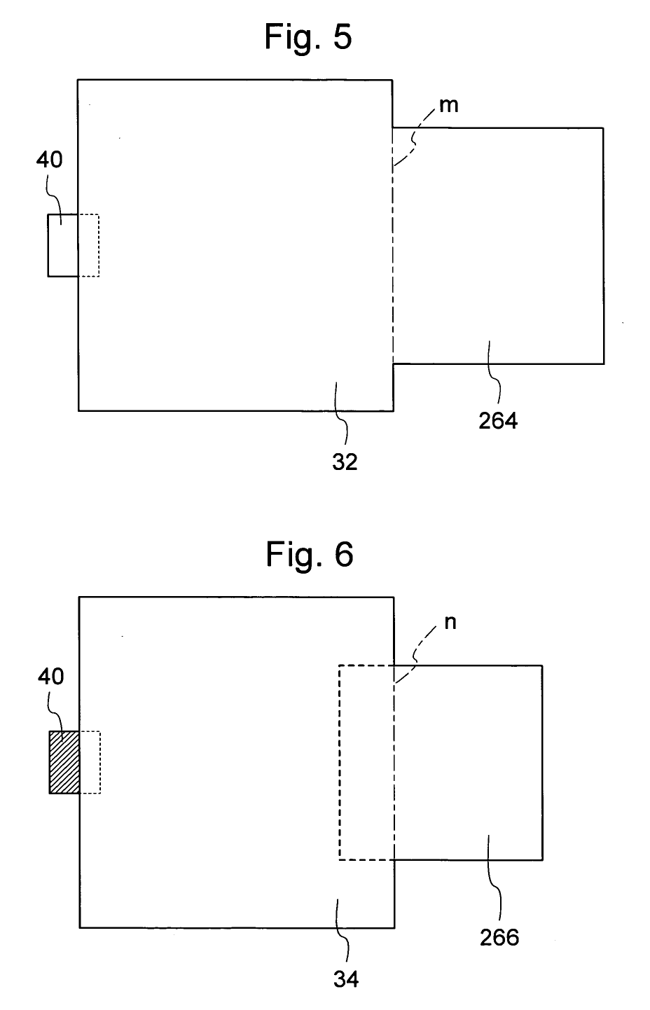

The sheet piece 260 may be a sheet piece 264 formed by extending a marginal part of the individual wrapping container 32 in part as is shown in FIG. 5, or may be a sheet piece 266 joined to the outer face of the individual wrapping container 34 as shown in FIG. 6. The sheet piece 264 may be folded inward the individual wrapping container 32 along the fold line “m”, and may be wrapped a part or whole of the interlabial pad. Alternatively, the sheet piece 266 may be folded inward the individual wrapping container 34 along the fold line “n”, and may be wrapped a part or whole of the interlabial pad. [Fourth embodiment]

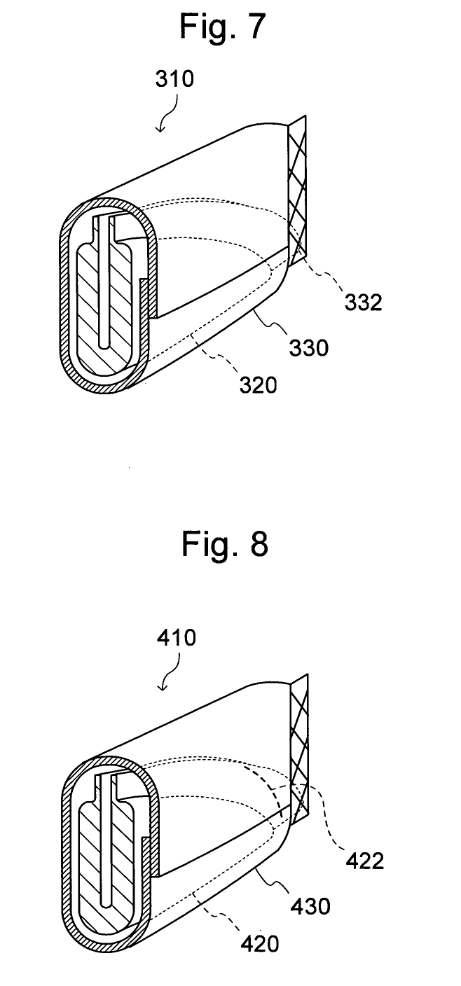

FIG. 7 depicts an end view of the wrapping body 310 according to fourth embodiment of the invention.

This embodiment is different from the above first embodiment in terms of the wrapping body 310 having a concavity and convexity part 332 by way of engaging emboss as a connecting means for connecting a part of the individual wrapping container 330 to a part of the interlabial pad 320. Accordingly, displacement of the interlabial pad 320 included in the individual wrapping container 330 is prevented by binding in the packed state, therefore, shape of the interlabial pad 320 within the individual wrapping container 330 can be preserved.

[Fifth embodiment]

FIG. 8 depicts an end view of the wrapping body 410 according to fifth embodiment of the invention.

In this embodiment, the wrapping body 410 has a concavity and convexity part 432 as a connecting means similarly to the wrapping body 310 of the fourth embodiment, as shown in FIG. 8. This embodiment is different from the above first embodiment in terms of the wrapping body 410, a part of the interlabial pad 420 being joined to a part of the individual wrapping container 430 by the concavity and convexity part 432. Accordingly, displacement of the interlabial pad 420 included in the individual wrapping container 430 is prevented by the connecting means in the packed state, therefore, shape of the interlabial pad within the individual wrapping container can be preserved. Also, the wrapping body 410 has perforations 422 corresponding to a cutoff part provided between the interlabial pad 420 and the individual wrapping container 430. According to the wrapping body 410 of the invention, the interlabial pad 420 can be thereby cut to separate from the portion where the interlabial pad 420 and the individual wrapping container 430 are joined, by merely cutting off along the perforations 422. Therefore, the wearer can readily draw the interlabial pad 420 from the individual wrapping container 430 without any sense of resistance.

[Sixth Embodiment]

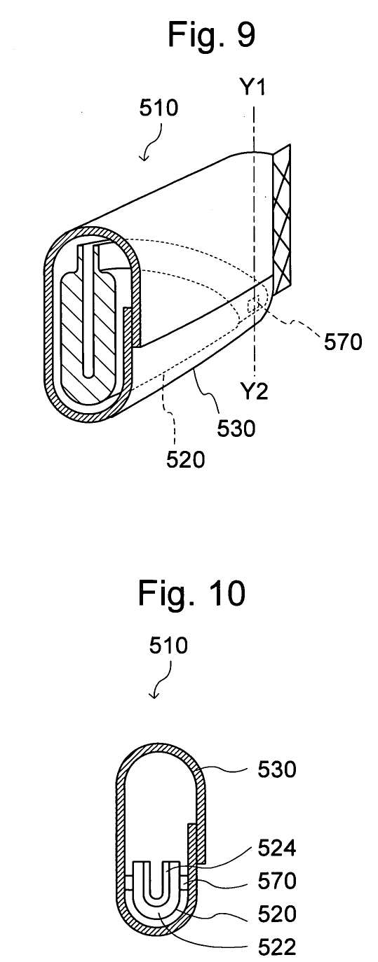

FIG. 9 is a perspective view showing the end view of the wrapping body 510 according to sixth embodiment of the invention. FIG. 10 depicts a cross sectional view in a direction of Y1-Y2 of the wrapping body 510 according to the sixth embodiment of the invention.

This embodiment is different from the above first embodiment in terms of application of an adhesive 570 as a connecting means, as shown in FIG. 9 and FIG. 10, between the interlabial pad 530 and the individual wrapping container 520 for connecting these, as a displacement preventive means. Because the adhesive 570 is applied, the displacement can be prevented. A surface side sheet 522 of the interlabial pad 520 to be brought into contact with the inner face of the individual wrapping container is an aggregate of fibers. Thus, when the interlabial pad 520 is drawn from the individual wrapping container 530, only a part of the fibers to which the adhesive 570 is adhered is cut to separate from the interiabial pad 520itself. Furthermore, the surface side sheet 522 of the interlabial pad 520 which comprises a fiber aggregate or a film material is subjected to a treatment for detachment by e.g., applying silicon or the like. Therefore, because of the adhesiveness not being so potent, the interlabial pad 520 can be drawn without damage thereto while leaving the adhesive 570 on the individual wrapping container 530 by pulling the interlabial pad 520 itself. In connection with the place where the adhesive 570 is applied, it is preferably applied to a region exposed out from the labia pudendi, taking into account of the foreign body sensation to the wearer.

<Process For Measuring Coefficient of Static Friction of Each Material Constituting Wrapping Body>

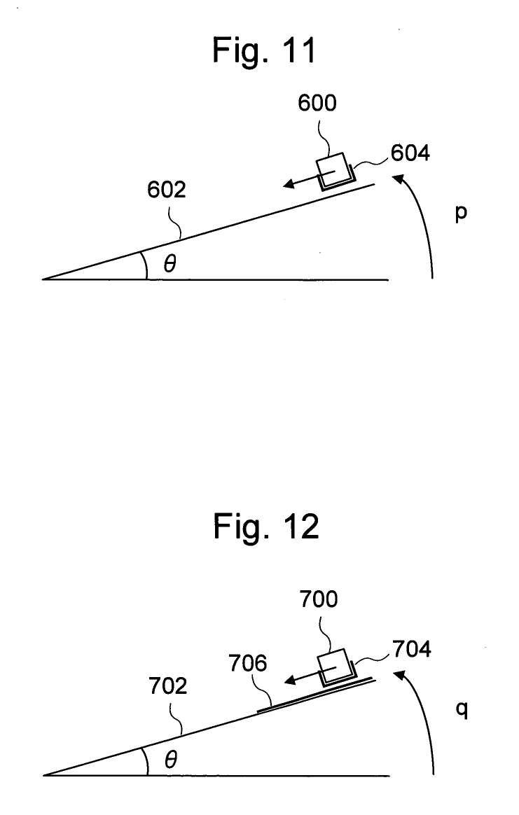

Process for measuring coefficient of static friction of each material constituting the wrapping body of the invention is illustrated in FIG. 11. First, a material 604 constituting the wrapping body (surface side sheet and back face side sheet, and individual wrapping container) is taken each having a length in the longitudinal direction of the absorbent element of the interlabial pad (hereinafter, referred to as “MD direction”) being 15 mm, and having a length in the cross direction which is orthogonal to the longitudinal direction of the absorbent element of the interlabial pad (hereinafter, referred to as “CD direction”) being 15 mm. Next, thus taken material is mounted to a block 600 of 10 g/(10 mm×10 mm) to render the measuring face of the taken material to be a friction face.

Subsequently, the block 600 to which the material 604 constituting the wrapping body (surface side sheet and back face side sheet, and individual wrapping container) is mounted is placed on a slip tester 602 in the form of a glass plate. Thereafter, the slip tester 602 is inclined in a direction represented by the symbol “p”, and the angle of inclination at a moment when the block 600 started to slip is measured.

Finally, the coefficient of static friction is determined. When the coefficient of static friction of each material 604 constituting the wrapping body is represented by μ, and the angle of inclination at a moment when the block 600 starts to slip is represented by θ, the following formula is derived.
μ=tan θ [Mathematical formula 1]

According to the above formula, the coefficient of static friction of each element 604 constituting the wrapping body can be determined.

<Process for Measuring Coefficient of Static Friction Between Interlabial Pad and Individual Wrapping Container>

Process for measuring coefficient of static friction between the interlabial pad and the individual wrapping container of the invention is illustrated in FIG. 12. First, outer face 704 of the interlabial pad (surface side sheet and back face side sheet) is taken having a length in the longitudinal direction of the absorbent element of the interlabial pad (hereinafter, referred to as “MD direction”) being 15 mm, and having a length in the cross direction which is orthogonal to the longitudinal direction of the absorbent element of the interlabial pad (hereinafter, referred to as “CD direction”) being 15 mm, respectively. Next, thus taken material is mounted to a block 700 of 10 g/(10 mm×10 mm) to render the measuring face of the taken material to be a friction face. Subsequently, the individual wrapping container is taken to give at least MD×CD:15 mm×15 mm or greater. Further, the inner face 706 of the individual wrapping container as a friction face is mounted to a slip tester 702 in the form of a glass plate without any gap.

Subsequently, the block 700 to which the outer face 704 of the interlabial pad (surface side sheet and back face side sheet) is mounted is placed on a slip tester 702 to which the inner face 706 of the individual wrapping container is mounted without any gap. Thereafter, the slip tester 702 is inclined in a direction represented by the symbol q, and the angle of inclination at a moment when the block 700 starts to slip is measured.

Finally, the coefficient of static friction is determined. When the coefficient of static friction between the outer face 704 of the interlabial pad and the inner face 704 of the individual wrapping container is represented by μ, and the angle of inclination at a moment when the block 700 starts to slip is represented by θ, the following formula is derived.
μ=tan θ [Mathematical formula 2]

According to the above formula, the coefficient of static friction between the outer face of the interlabial pad and the inner face of the individual wrapping container can be determined.

[Material Constituting Interlabial Pad]

<Individual Wrapping Container>

Examples of the material of the individual wrapping container include polyethylene, polypropylene, polyethylene terephthalate, polyvinyl alcohol, polylactic acid, polybutyl succinate, or nonwoven fabrics, paper, and laminate materials thereof having a thickness of 15 to 60 μm. Alternatively, a breathable film obtained by mixing an inorganic filler to a resin and drawing the mixture may be used. Specifically, examples include films prepared using a low density polyethylene (LDPE) resin as a main component and adjusted to give a quantity per unit area in a range from 15 to 35 g/m² with fine convexities provided thereon. Height of the fine convexity is in a range from 0.05 mm to 2 mm, and interval between the fine convexities is in a range from 0.3 mm to 20 mm. Among them, films prepared using a low density polyethylene (LDPE) resin as a main component to have fine convexities arranged with the height of the convexity being 0.2 mm, the interval therebetween being 1.5 mm and the shape being staggered have a coefficient of static friction of 0.250. Further, a rubber component which can provide an elastomer characteristic, an anti-slip agent or the like may be admixed together with the low density polyethylene (LDPE) resin. Alternatively, examples include materials for individual wrapping container prepared by spraying the melt blown on a film comprising polyethylene, polypropylene or the like, or a nonwoven fabric or paper to have a melt blown surface on the inner face of the individual wrapping container. Because the melt blown is an aggregate of very fine fibers, a great contact area is achieved. Accordingly, the coefficient of static friction falling within a suitable range can be achieved. In addition, it is preferred that the individual wrapping container can block the color of the bodily fluid absorbed in the interlabial pad. Thus, a pigment may be admixed in a range from 0.2 to 10%, or printing with an ink or the like may be performed. Also, examples of the individual wrapping container having an air permeability of a range from 0.040 KPa·s/m to 1.000 KPa·s/m include breathable films obtained by mixing an inorganic filler to a resin and drawing the mixture; or films having communicating pores prepared by partially subjecting to a treatment for opening pores; and polyethylene, polypropylene, polyethylene terephthalate, polyvinyl alcohol, polylactic acid, polybutyl succinate, or nonwoven fabrics, paper, and laminate materials thereof with communicating pores provided therein having a thickness of 15 to 60 μm. Specifically, so called SMS nonwoven fabrics are included constituted from spun bond/melt blown/spun bond with the weight of 6 to 10 gsm/2 to 20 gsm/6 to 10 gsm. Because the melt blown is an aggregate of very fine fibers, it has a high density and excellent barrier property. Therefore, adequate air permeability is achieved by adjusting the quantity per unit area or the like of the melt blown layer.

<Surface Side Sheet of Interlabial Pad>

For the surface side sheet 22 disposed to the body side of the interlabial pad, a material which is water permeable, liquid-philic and nonirritating to the skin may be used. Examples of such a material include pulp, chemical pulp, rayon, cotton, acetate, a synthetic fiber, physically embossed rayon or acetate, chemical pulp crosslinked with a crosslinking agent followed by crimping, which may be used alone or in combination. Examples of the method of the production of the sheet include a spun bond method, a point bond method, an air through method, a melt blown method, a spun lace method, a paper making method and the like. Among such methods of the production, the spun lace method is preferred in which fiber confounding is executed by hydroentanglement, thereby capable of eliminating an oil solution attached to the fiber by the water flow, because any oil solution, lubricant or the like, which may elevate the slipping property with the inner face of the individual wrapping container, is not preferably included in the outer face of the interlabial pad.

<Back Face Side Sheet of Interlabial Pad>

For the back face sheet 24 which is liquid impermeable for use in the interlabial pad, a water impermeable material may be used. As the material for this water impermeable sheet, any one which can prevent the leakage of the menstrual blood retained in the absorptive member outside of the interlabial pad may be used. In addition, through the use of a moisture permeable material, retention of moisture during application can be reduced, thereby enabling decrease of sense of discomfort during application. Examples of such material of a back face side sheet include polyethylene, polypropylene, polyethylene terephthalate, polyvinyl alcohol, polylactic acid, polybutyl succinate, or nonwoven fabrics, paper, and laminate materials thereof having a thickness of 15 to 60 μm. Alternatively, a breathable film obtained by mixing an inorganic filler to a resin and drawing the mixtuermay be also used. Specifically, examples include films prepared using a low density polyethylene (LDPE) resin as a main component and adjusted to give a quantity per unit area in a range from 15 to 35 g/m² with fine convexities provided thereon. Height of the fine convexity is in a range from 0.05 mm to 2 mm, and interval between the fine convexities is in a range from 0.3 mm to 20 mm. Among them, films prepared using a low density polyethylene (LDPE) resin as a main component to have fine convexities arranged with the height of the convexity being 0.2 mm, the interval therebetween being 1.5 mm and the shape being staggered have a coefficient of static friction of 0.250. Further, a rubber component which can provide an elastomer characteristic, an anti-slip agent or the like may be admixed together with the low density polyethylene (LDPE) resin. Alternatively, examples include back face side sheets prepared by spraying the melt blown on a film comprising polyethylene, polypropylene or the like, or a nonwoven fabric or paper to have a melt blown surface on the outer face of the interlabial pad. Because the melt blown is an aggregate of very fine fibers, a great contact area is achieved. Accordingly, the coefficient of static friction falling within a suitable range can be achieved.

The present invention can be utilized as an individual wrapping container and an interlabial pad not causing shifting of the internally included interlabial pad from its proper position on the individual wrapping container.

1. An Individual wrapping container for individually wrapping an interlabial pad applied by inserting into wearer's labia; wherein
the individual wrapping container comprises an inner face having a region to be brought into contact with the interlabial pad when the interlabiel pad is included therein, and an outer face to be the reverse face of the inner face; and wherein
the inner face comprising a displacement preventive means to bind the interlabial pad at a predetermined position thereby preventing the displacement of the interlabial pad.

2. The individual wrapping container according to claim 1, wherein the displacement preventive means has a shifting resistant element having a coefficient of static friction of a range from 0.200 to 0.600 disposed on the entire face of the inner face.

3. The individual wrapping container according to claim 1, wherein the displacement preventive means has a shifting resistant element having a coefficient of static friction of a range from 0.200 to 0.600 disposed on a part of the inner face corresponding to the entirety of the predetermined position.

4. The individual wrapping container according to claim 1, wherein the displacement preventive means has a shifting resistant element having a coefficient of static friction of a range from 0.200 to 0.600 disposed on the entire face of the inner face, or on a part of the inner face corresponding to a part of the predetermined position.

5. The individual wrapping container according to claim 1, wherein the displacement preventive means is a breathable material constituting the aforementioned individual wrapping container, and the air permeability of the breathable material is a range form 0.040 KPa·s/m to 1.000 KPa·s/m.

6. The individual wrapping container according to claim 1, wherein the displacement preventive means is a sheet piece formed by being mounted on the inner face; and wherein
the sheet piece has length and flexibility enabling to wind around the interlabial pad, and has a release part permitting easy release of the winding when the interlabial pad is drawn from the individual wrapping container.

7. The individual wrapping container according to claim 1, wherein the displacement preventive means is a sheet piece formed by being mounted on the outer face; and wherein
the sheet piece has length and flexibility enabling to wind around the interlabial pad, and has a release part permitting easy release of the winding when the interlabial pad is drawn from the individual wrapping container.

8. The individual wrapping container according to claim 1, wherein the displacement preventive means is a sheet piece formed by extending a part of a material that constitutes the individual wrapping container; and wherein
the sheet piece has length and flexibility enabling to wind around the interlabial pad, and has a release part permitting easy release of the winding when the interlabial pad is drawn from the a individual wrapping container.

9. The individual wrapping container according to claim 6, wherein the individual wrapping container has an opening part for opening the individual wrapping container, and wherein
the release part of the sheet piece is disposed in the vicinity of the opening part of the individual wrapping container.

10. An interlabial pad applied by inserting into wearer's labia, which comprises a surface side sheet orientated to a body face side and a back face side sheet orientated to the reverse side face of the body face side, wherein
the surface side sheet has a displacement preventive means for the surface side sheet has a displacement preventive means for preventing shifting from a predetermined position on the individual wrapping container where the interlablal pad is disposed when the interlabial pad is packed in an individual wrapping container.

11. An interlabial pad applied by inserting into wearer's labia, which comprises a surface side sheet orientated to a body face side and a back face side sheet orientated to the reverse side face of the body face side, wherein
the back face side sheet has a displacement preventive means for preventing shifting from a predetermined position on the individual wrapping container where the interlabial pad is disposed when the interlabial pad is packed in an individual wrapping container.

12. An interlabial pad applied by inserting into wearer's labia, which comprises a surface side sheet orientated to a body face side and a back face side sheet orientated to the reverse side face of the body face side, wherein
the surface side sheet and the back face side sheet have a displacement preventive means for preventing shifting from a predetermined position on the individual wrapping container where the interlabial pad is disposed when the interlabial pad is packed in an individual wrapping container.

13. The interlabial pad according to claim 10, wherein the displacement preventive means of the interlabial pad comprises a shifting resistant element having a coefficient of static friction of a range from 0.200 to 0.600.

14. A wrapping body which comprises an interlabial pad applied by inserting into wearer's labia, and an individual wrapping container for individually wrapping the interlabial pad, wherein
a coefficient of static friction of a part where the interlabial pad is in contact with the individual wrapping container is in a range from 0.220 to 0.500.

15. A wrapping body which comprises an interlabial pad applied by inserting into wearer's labia, and an individual wrapping container for individually wrapping the interlabial pad, wherein a connecting means for connecting a part of the interlabial pad to a part of the individual wrapping container for preventing a displacement of the interlabial pad by binding the interlabial pad at a predetermined position on the individual wrapping container.

16. The wrapping body according to claim 15, wherein the connecting means integrally connects a part of the individual wrapping container to a part of the interlabial pad.

17. The wrapping body according to claim 15, wherein a part connected by the connecting means or the vicinity thereof has a cutoff part to be cut off when the interlabial pad is drawn from the individual wrapping container.

18. The wrapping body according to claim 15, wherein the connecting means is an adhesive applied for adhering the interlabial pad.

19. The wrapping body according to claim 15, wherein the connecting means is an adhesive applied for adhering the individual wrapping container.

20. The wrapping body according to claim 15, wherein the connecting means is an adhesive applied for adhering the interlabial pad and the individual wrapping container.

21. The individual wrapping container according to claim 7, wherein the individual wrapping container has an opening part for opening the individual wrapping container, and wherein
the release part of the sheet piece is disposed in the vicinity of the opening part of the individual wrapping container.

22. The individual wrapping container according to claim 8, wherein the individual wrapping container has an opening part for opening the individual wrapping container, and wherein
the release part of the sheet piece is disposed in the vicinity of the opening part of the individual wrapping container.

23. The interlabial pad according to claim 11, wherein the displacement preventive means of the interlabial pad comprises a shifting resistant element having a coefficient of static friction of a range from 0.200 to 0.600.
